(19) United States
(12) Patent Application Publication
(10) Pub. No.: US 2014/0031859 A1
(43) Pub. Date: Jan. 30, 2014

(54) Circular Wound Compress

(71) Applicant: Regina Jacqueline D'Andrea, Providence, RI (US)

(72) Inventor: Regina Jacqueline D'Andrea, Providence, RI (US)

(21) Appl. No.: 13/356,287

(22) Filed: Jan. 23, 2012

(51) Int. Cl. A61B 17/132

(52) U.S. Cl. CPC A61B 17/1322
USPC 606/203

(57) ABSTRACT

I believe I am the original and first inventor of the subject matter which is claimed for which patent is sought; am a citizen of the United States of America; have reviewed and understand the contents of the application, including the claims, as amended by any amendment specifically referred to in the oath or declaration; acknowledge the duty to disclose to the Office all information known to the person to be material to patentability as defined in §1.56.

### CROSS-REFERENCE

| Patent # | Date Filed | Name of Inventors |
|---|---|---|
| 2,383,670 | Jul. 1, 1942 | Chester C. Moss, Jackson, Mich., assignor |
| | | to Walker Manufacturing Company of |
| | | Wisconsin, Racine, Wis., a corporation of |
| | | Wisconsin. |
| 1,597,525 | Jan. 11, 1924 | Arthur H. Knake, of St. James, Minnesota. |

### STATEMENT REGARDING FEDERALLY SPONSORED RESEARCH OR DEVELOPMENT.

There has been no federally sponsored research of development involved in the development of this design patent.

### DESCRIPTION OF THE FIGURE(S) OF THE DRAWING

FIG. 1 is a frontal view of the circular disk 1 of the circular wound compress.

FIG. 2 is side view of the circular disk 1 of the circular wound compress.

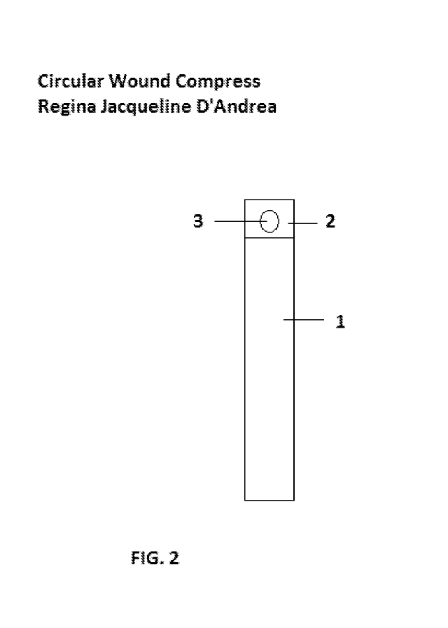

FIG. 3 is a frontal view of the circular wound compress in operation.

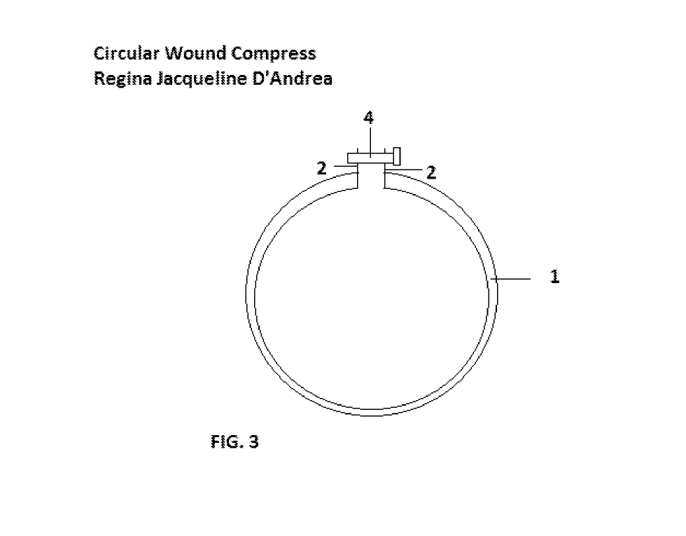

### DETAILED DESCRIPTION OF THE PREFERRED EMBODIMENT

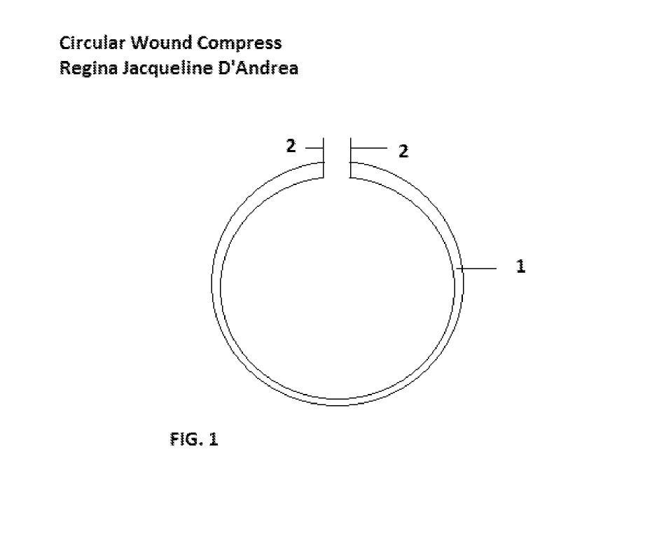

Referring to FIG. 1, a circular disk 1 is preferably formed of a hard material such as plastic or metal and may come in a different shape, dimensions, or thickness to fit the body part being treated. The circular disk 1 has an opening with plates 2 that come up on each side of the opening, also preferably made of a hard material such as metal or plastic. Each of these plates 2 must have screw holes 3 in identical places.

In operation, the circular disk 1 is to be placed directly on top of the wound. Screws 4 are then screwed through the screw holes 3 so that the circular disk is held securely with the affected body part within the circular disk 1. The screws may be tightened to achieve the degree of pressure required, or loosened to relieve pressure and increase circulation to the body part affected by the wound.

While the invention is susceptible to various modifications and alternative forms, specific embodiments thereof have been shown in the drawings and are herein described in detail. It should be understood, however, that the invention is not to be limited to the particular form disclosed, but to the contrary, the invention is to cover all modifications, equivalents, and alternatives falling within the spirit and scope of the appended claims.

### Feature Description

### Field of Invention

The present invention relates to applying pressure to bleeding wounds to stop them from bleeding.

### Background of the Invention

Bleeding wounds require the application of constant pressure to stop bleeding. Some bleeding may be stopped with stitches but sometimes stitches are not an option. For example, doctors usually don't use stitches to close a wound after 8 hours because there is a chance they may seal in infection. Another example is if a patient is performing at-home care. Another example is if the wound is too large to be stitched. Stitches may be avoided if pressure stops bleeding within fifteen to twenty minutes. Currently, pressure is applied to dressed wounds by wrapping a pressure bandage around the wound. The process of wrapping the bandage tightly can be irritating to the wound site because the pulling of the bandage pulls on the wound, therefore preventing healing and possibly increasing bleeding. This also prevents the bandage from being applied tightly enough, which reduces pressure and reduces healing. The wound would benefit from a mechanism that would maximize pressure directly on the wound without pulling on the wound.

### Summary of the Invention

The circular disk of the circular wound compress is placed directly on top of the wound. Screws are screwed through screw holes so the circular disk is held in place with the body part within the circular disk. The screws may be tightened to increase pressure on the wound or loosened to alleviate pressure and allow more circulation.

Other aspects and features of the present invention will become apparent from consideration of the following description taken in conjunction with the accompanying drawings.

What is claimed:

1. A circular wound compress comprises a circular disk 1 with an opening with plates 2 on either side of the opening with screw holes 3 and a screw 4 to hold the circular disk 1 in place.

2. The device of claim 1, wherein there is padding or absorbent material such as batting material, foam padding, cotton, gauze, and the like, in between or around the two blocks.

3. The device of claim 1, wherein the plates 2 have multiple screw holes 3 and multiple screws 4.

4. The device of claim 1, wherein washers are places between the screws and the blocks.

5. The device of claim 1, wherein another binding mechanism such as a clamp, elastic band, nail, and the like, is used with or without the screws.
